SEQUENCE LISTING (79 sequences)

SEQ ID NO: 1 - DNA, 735 bp, Artificial Sequence
  Synthetic construct
gagctcgtgctgacccagactccatcccccgtgtctgcagttgtgggaggcacagtcaccatcaattgccagtccagtcagagtgtttggggtaacaaccgcttatcctggtatcagcagaaaccagggcagcctcccaggctcctaatgtattatgcatccaatctggcatctggggtctcatcgcggttcaaaggcagtggatctgggacacaattcactctcaccatcagcgacgtgcagtgtgacgatgctgccacttactactgtcaaggcggatttgagtgtagtggtggtgattgtgttggtttcggcggagggaccgagctggagatcctaggtggttcctctagatcttcctcctctggtggcggtggctcgggcggtggtgggcagtcggtggaggagtccgggggtcgcctggtagcgcctggaggatccctgacactcacctgcacagtctctggaatcgacctcagtagcgatgcaatgagctgggtccgccaggctccagggaaggggctggaatggatcggaacgatttatggtagtgctggcacatactacgcgacctgggcgaaaggccgattcaccatctccaaaacctcgaccacggtggatctgaaaatgaccagtctgacaaccgaggacacggccacctatttctgtaccagagcttttagcaacactcgattggatctctggggccagggcaccctggtcaccatctcctca

SEQ ID NO: 2 - protein, 245 aa, Artificial Sequence
  Synthetic construct
ELVLTQTPSPVSAVVGGTVTINCQSSQSVWGNNRLSWYQQKPGQPPRLLMYYASNLASGVSSRFKGSGSGTQFTLTISDVQCDDAATYYCQGGFECSGGDCVGFGGGTELEILGGSSRSSSSGGGGSGGGGQSVEESGGRLVAPGGSLTLTCTVSGIDLSSDAMSWVRQAPGKGLEWIGTIYGSAGTYYATWAKGRFTISKTSTTVDLKMTSLTTEDTATYFCTRAFSNTRLDLWGQGTLVTISS

SEQ ID NO: 3 - DNA, 336 bp, Artificial Sequence
  Synthetic construct
gtggaggagtccgggggtcgcctggtagcgcctggaggatccctgacactcacctgcacagtctctggaatcgacctcagtagcgatgcaatgagctgggtccgccaggctccagggaaggggctggaatggatcggaacgatttatggtagtgctggcacatactacgcgacctgggcgaaaggccgattcaccatctccaaaacctcgaccacggtggatctgaaaatgaccagtctgacaaccgaggacacggccacctatttctgtaccagagcttttagcaacactcgattggatctctggggccagggcaccctggtcaccatctcctca

SEQ ID NO: 4 - protein, 112 aa, Artificial Sequence
  Synthetic construct
VEESGGRLVAPGGSLTLTCTVSGIDLSSDAMSWVRQAPGKGLEWIGTIYGSAGTYYATWAKGRFTISKTSTTVDLKMTSLTTEDTATYFCTRAFSNTRLDLWGQGTLVTISS

SEQ ID NO: 5 - DNA, 339 bp, Artificial Sequence
  Synthetic construct
gagctcgtgctgacccagactccatcccccgtgtctgcagttgtgggaggcacagtcaccatcaattgccagtccagtcagagtgtttggggtaacaaccgcttatcctggtatcagcagaaaccagggcagcctcccaggctcctaatgtattatgcatccaatctggcatctggggtctcatcgcggttcaaaggcagtggatctgggacacaattcactctcaccatcagcgacgtgcagtgtgacgatgctgccacttactactgtcaaggcggatttgagtgtagtggtggtgattgtgttggtttcggcggagggaccgagctggagatccta

SEQ ID NO: 6 - protein, 113 aa, Artificial Sequence
  Synthetic construct
ELVLTQTPSPVSAVVGGTVTINCQSSQSVWGNNRLSWYQQKPGQPPRLLMYYASNLASGVSSRFKGSGSGTQFTLTISDVQCDDAATYYCQGGFECSGGDCVGFGGGTELEIL

SEQ ID NO: 7 - DNA, 15 bp, Artificial Sequence
  Synthetic construct
agcgatgcaatgagc

SEQ ID NO: 8 - protein, 5 aa, Artificial Sequence
  Synthetic construct
SDAMS

SEQ ID NO: 9 - DNA, 45 bp, Artificial Sequence
  Synthetic construct
acgatttatggtagtgctggcacatactacgcgacctgggcgaaa

SEQ ID NO: 10 - protein, 15 aa, Artificial Sequence
  Synthetic construct
TIYGSAGTYYATWAK

SEQ ID NO: 11 - DNA, 27 bp, Artificial Sequence
  Synthetic construct
gcttttagcaacactcgattggatctc

SEQ ID NO: 12 - protein, 9 aa, Artificial Sequence
  Synthetic construct
AFSNTRLDL

SEQ ID NO: 13 - DNA, 18 bp, Artificial Sequence
  Synthetic construct
cagtccagtcagagtgtt

SEQ ID NO: 14 - protein, 6 aa, Artificial Sequence
  Synthetic construct
QSSQSV

SEQ ID NO: 15 - DNA, 21 bp, Artificial Sequence
  Synthetic construct
tatgcatccaatctggcatct

SEQ ID NO: 16 - protein, 7 aa, Artificial Sequence
  Synthetic construct
YASNLAS

SEQ ID NO: 17 - DNA, 39 bp, Artificial Sequence
  Synthetic construct
caaggcggatttgagtgtagtggtggtgattgtgttggt

SEQ ID NO: 18 - protein, 13 aa, Artificial Sequence
  Synthetic construct
QGGFECSGGDCVG

SEQ ID NO: 19 - DNA, 753 bp, Artificial Sequence
  Synthetic construct
gagctcgatatgacccagactccagcctctgtggaggtagctgtgggaggcacagtcaccatcaattgccaggccagtcagagtgtttataaggacaacaacttagcctggtatcagcagaaaccagggcagcctcccaagctcctgatctacaaggcttccactctggcatctggggtcccgtcgcggttcaaaggcagtggatctgggacacagttcactctcaccatcagcgacgtgcagtgtgacgatgctaccacttactactgtgcaggcggttggaattctaatgatgatacgtttgctttcggcggagggaccgcggtggtggtcagaggtggttcctctagatcttcctcctctggtggcggtggctcgggcggtggtgggcagtcgctggaggagtccgggggtcgcctggtcacgcctgggacacccctgacactcacctgcacagtctctggattctccctcagtaactactggatgaactgggtccgccaggctccagggaagggactggaatggatcggagtcattagtaggagtggtatcacaggctacgcgagctgggcgaaaggccgattcaccatctccaaaacctcgaccacagtggatctgaaaatcaccagtccgacaaccgaggacacggccacctatttctgtgccagacaatattattctggttatggtgatgttgcttatgttgactttaacttgtggggccaaggcaccctggtcaccatctcttca

SEQ ID NO: 20 - protein, 251 aa, Artificial Sequence
  Synthetic construct
ELDMTQTPASVEVAVGGTVTINCQASQSVYKDNNLAWYQQKPGQPPKLLIYKASTLASGVPSRFKGSGSGTQFTLTISDVQCDDATTYYCAGGWNSNDDTFAFGGGTAVVVRGGSSRSSSSGGGGSGGGGQSLEESGGRLVTPGTPLTLTCTVSGFSLSNYWMNWVRQAPGKGLEWIGVISRSGITGYASWAKGRFTISKTSTTVDLKITSPTTEDTATYFCARQYYSGYGDVAYVDFNLWGQGTLVTISS

SEQ ID NO: 21 - DNA, 357 bp, Artificial Sequence
  Synthetic construct
ctggaggagtccgggggtcgcctggtcacgcctgggacacccctgacactcacctgcacagtctctggattctccctcagtaactactggatgaactgggtccgccaggctccagggaagggactggaatggatcggagtcattagtaggagtggtatcacaggctacgcgagctgggcgaaaggccgattcaccatctccaaaacctcgaccacagtggatctgaaaatcaccagtccgacaaccgaggacacggccacctatttctgtgccagacaatattattctggttatggtgatgttgcttatgttgactttaacttgtggggccaaggcaccctggtcaccatctcttca

SEQ ID NO: 22 - protein, 119 aa, Artificial Sequence
  Synthetic construct
LEESGGRLVTPGTPLTLTCTVSGFSLSNYWMNWVRQAPGKGLEWIGVISRSGITGYASWAKGRFTISKTSTTVDLKITSPTTEDTATYFCARQYYSGYGDVAYVDFNLWGQGTLVTISS

SEQ ID NO: 23 - DNA, 336 bp, Artificial Sequence
  Synthetic construct
gagctcgatatgacccagactccagcctctgtggaggtagctgtgggaggcacagtcaccatcaattgccaggccagtcagagtgtttataaggacaacaacttagcctggtatcagcagaaaccagggcagcctcccaagctcctgatctacaaggcttccactctggcatctggggtcccgtcgcggttcaaaggcagtggatctgggacacagttcactctcaccatcagcgacgtgcagtgtgacgatgctaccacttactactgtgcaggcggttggaattctaatgatgatacgtttgctttcggcggagggaccgcggtggtggtcaga

SEQ ID NO: 24 - protein, 112 aa, Artificial Sequence
  Synthetic construct
ELDMTQTPASVEVAVGGTVTINCQASQSVYKDNNLAWYQQKPGQPPKLLIYKASTLASGVPSRFKGSGSGTQFTLTISDVQCDDATTYYCAGGWNSNDDTFAFGGGTAVVVR

SEQ ID NO: 25 - DNA, 15 bp, Artificial Sequence
  Synthetic construct
aactactggatgaac

SEQ ID NO: 26 - protein, 5 aa, Artificial Sequence
  Synthetic construct
NYWMN

SEQ ID NO: 27 - DNA, 45 bp, Artificial Sequence
  Synthetic construct
gtcattagtaggagtggtatcacaggctacgcgagctgggcgaaa

SEQ ID NO: 28 - protein, 15 aa, Artificial Sequence
  Synthetic construct
VISRSGITGYASWAK

SEQ ID NO: 29 - DNA, 48 bp, Artificial Sequence
  Synthetic construct
caatattattctggttatggtgatgttgcttatgttgactttaacttg

SEQ ID NO: 30 - protein, 16 aa, Artificial Sequence
  Synthetic construct
QYYSGYGDVAYVDFNL

SEQ ID NO: 31 - DNA, 18 bp, Artificial Sequence
  Synthetic construct
caggccagtcagagtgtt

SEQ ID NO: 32 - protein, 6 aa, Artificial Sequence
  Synthetic construct
QASQSV

SEQ ID NO: 33 - DNA, 21 bp, Artificial Sequence
  Synthetic construct
aaggcttccactctggcatct

SEQ ID NO: 34 - protein, 7 aa, Artificial Sequence
  Synthetic construct
KASTLAS

SEQ ID NO: 35 - DNA, 36 bp, Artificial Sequence
  Synthetic construct
gcaggcggttggaattctaatgatgatacgtttgct

SEQ ID NO: 36 - protein, 12 aa, Artificial Sequence
  Synthetic construct
AGGWNSNDDTFA

SEQ ID NO: 37 - DNA, 42 bp, Artificial Sequence
  Synthetic construct
gctgcccaaccagccatggcccagtcggtggaggagtccrgg

SEQ ID NO: 38 - DNA, 42 bp, Artificial Sequence
  Synthetic construct
gctgcccaaccagccatggcccagtcggtgaaggagtccgag

SEQ ID NO: 39 - DNA, 42 bp, Artificial Sequence
  Synthetic construct
gctgcccaaccagccatggcccagtcgytggaggagtccggg

SEQ ID NO: 40 - DNA, 44 bp, Artificial Sequence
  Synthetic construct
gctgcccaaccagccatggcccagsagcagctgrtggagtccgg

SEQ ID NO: 41 - DNA, 45 bp, Artificial Sequence
  Synthetic construct
cgatgggcccttggtggaggctgargagayggtgaccagggtgcc

SEQ ID NO: 42 - DNA, 38 bp, Artificial Sequence
  Synthetic construct
gggcccaggcggccgagctcgtgmtgacccagactcca

SEQ ID NO: 43 - DNA, 38 bp, Artificial Sequence
  Synthetic construct
gggcccaggcggccgagctcgatmtgacccagactcca

SEQ ID NO: 44 - DNA, 38 bp, Artificial Sequence
  Synthetic construct
gggcccaggcggccgagctcgtgatgacccagactgaa

SEQ ID NO: 45 - DNA, 40 bp, Artificial Sequence
  Synthetic construct
gggcccaggcggccgagctcgtgctgactcagtcgccctc

SEQ ID NO: 46 - DNA, 42 bp, Artificial Sequence
  Synthetic construct
agatggtgcagccacagttcgtttgatttccacattggtgcc

SEQ ID NO: 47 - DNA, 42 bp, Artificial Sequence
  Synthetic construct
agatggtgcagccacagttcgtaggatctccagctcggtccc

SEQ ID NO: 48 - DNA, 42 bp, Artificial Sequence
  Synthetic construct
agatggtgcagccacagttcgtttgacsaccacctcggtccc

SEQ ID NO: 49 - DNA, 45 bp, Artificial Sequence
  Synthetic construct
agatggtgcagccacagttcggcctgtgacggtcagctgggtccc

SEQ ID NO: 50 - DNA, 24 bp, Artificial Sequence
  Synthetic construct
gcctccaccaagggcccatcggtc

SEQ ID NO: 51 - DNA, 21 bp, Artificial Sequence
  Synthetic construct
agaagcgtagtccggaacgtc

SEQ ID NO: 52 - DNA, 24 bp, Artificial Sequence
  Synthetic construct
cgaactgtggctgcaccatctgtc

SEQ ID NO: 53 - DNA, 21 bp, Artificial Sequence
  Synthetic construct
ggccatggctggttgggcagc

SEQ ID NO: 54 - DNA, 21 bp, Artificial Sequence
  Synthetic construct
gctgcccaaccagccatggcc

SEQ ID NO: 55 - DNA, 41 bp, Artificial Sequence
  Synthetic construct
gaggaggaggaggaggaggcggggcccaggcggccgagctc

SEQ ID NO: 56 - DNA, 39 bp, Artificial Sequence
  Synthetic construct
gaggaggaggaggaggagagaagcgtagtccggaacgtc

SEQ ID NO: 57 - DNA, 54 bp, Artificial Sequence
  Synthetic construct
ggtggttcctctagatcttcctcctctggtggcggtggctcgggcggtggtggg

SEQ ID NO: 58 - protein, 18 aa, Artificial Sequence
  Synthetic construct
GGSSRSSSSGGGGSGGGG

SEQ ID NO: 59 - protein, 16 aa, Artificial Sequence
  Synthetic construct
PGSAAPYLKTKGGGSC

SEQ ID NO: 60 - protein, 16 aa, Artificial Sequence
  Synthetic construct
RNPGFYVEANPGGGSC

SEQ ID NO: 61 - protein, 16 aa, Artificial Sequence
  Synthetic construct
PHFPQFSYSASGGGSC

SEQ ID NO: 62 - DNA, 756 bp, Artificial Sequence
  Synthetic construct
gagctcgtgatgacccagactccatcgcccgtgtctgcagctgtgggaagcacagctaccatcaattgccaggccagtcagactgtttatagtaacgacttagcctggtttcagcagaaaccagggcagcctcccaagcgcctgatccgttctgcatccactctggcatctggggtcccgtcgcggttcaaaggcagtggatctgggacacagttcactctcaccatcagcgacgtgcagtgtgacgatgctgccacttattactgtctaggcgcttgggattgtcgtagtgctgattgttttgctttcggcggagggaccgaggtggtggtcaaatcctctggtggcggtgtttcgggcggtggtgggggtggttcctctagatcttcccagtcggtggaggagtccgggggtcgcctggtcacgcctgggacacccctgacactcacctgcacagtctctggattctccctcagtagctatgcaatgaactgggtccgccaggctccagggaaggggctggagtggatcggaatcattaatactgatggtagcgcatactacgcgacctgggcgaagcgccgattcaccatctccaaaacctcgaccacggtggatctgaaaatcaccagtctgacaaccgaggacacggccacttatttctgtggcagcggtagcgcgtggggcccaggcaccctggtcaccgtctccttagggcaacctaaggctccgtcagtcactagtggccaggccggc

SEQ ID NO: 63 - protein, 252 aa, Artificial Sequence
  Synthetic construct
ELVMTQTPSPVSAAVGSTATINCQASQTVYSNDLAWFQQKPGQPPKRLIRSASTLASGVPSRFKGSGSGTQFTLTISDVQCDDAATYYCLGAWDCRSADCFAFGGGTEVVVKSSGGGVSGGGGGGSSRSSQSVEESGGRLVTPGTPLTLTCTVSGFSLSSYAMNWVRQAPGKGLEWIGIINTDGSAYYATWAKRRFTISKTSTTVDLKITSLTTEDTATYFCGSGSAWGPGTLVTVSLGQPKAPSVTSGQAG

SEQ ID NO: 64 - DNA, 360 bp, Artificial Sequence
  Synthetic construct
gtggaggagtccgggggtcgcctggtcacgcctgggacacccctgacactcacctgcacagtctctggattctccctcagtagctatgcaatgaactgggtccgccaggctccagggaaggggctggagtggatcggaatcattaatactgatggtagcgcatactacgcgacctgggcgaagcgccgattcaccatctccaaaacctcgaccacggtggatctgaaaatcaccagtctgacaaccgaggacacggccacttatttctgtggcagcggtagcgcgtggggcccaggcaccctggtcaccgtctccttagggcaacctaaggctccgtcagtcactagtggccaggccggc

SEQ ID NO: 65 - protein, 120 aa, Artificial Sequence
  Synthetic construct
VEESGGRLVTPGTPLTLTCTVSGFSLSSYAMNWVRQAPGKGLEWIGIINTDGSAYYATWAKRRFTISKTSTTVDLKITSLTTEDTATYFCGSGSAWGPGTLVTVSLGQPKAPSVTSGQAG

SEQ ID NO: 66 - DNA, 336 bp, Artificial Sequence
  Synthetic construct
gagctcgtgatgacccagactccatcgcccgtgtctgcagctgtgggaagcacagctaccatcaattgccaggccagtcagactgtttatagtaacgacttagcctggtttcagcagaaaccagggcagcctcccaagcgcctgatccgttctgcatccactctggcatctggggtcccgtcgcggttcaaaggcagtggatctgggacacagttcactctcaccatcagcgacgtgcagtgtgacgatgctgccacttattactgtctaggcgcttgggattgtcgtagtgctgattgttttgctttcggcggagggaccgaggtggtggtcaaa

SEQ ID NO: 67 - protein, 112 aa, Artificial Sequence
  Synthetic construct
ELVMTQTPSPVSAAVGSTATINCQASQTVYSNDLAWFQQKPGQPPKRLIRSASTLASGVPSRFKGSGSGTQFTLTISDVQCDDAATYYCLGAWDCRSADCFAFGGGTEVVVK

SEQ ID NO: 68 - DNA, 15 bp, Artificial Sequence
  Synthetic construct
agctatgcaatgaac

SEQ ID NO: 69 - protein, 5 aa, Artificial Sequence
  Synthetic construct
SYAMN

SEQ ID NO: 70 - DNA, 45 bp, Artificial Sequence
  Synthetic construct
atcattaatactgatggtagcgcatactacgcgacctgggcgaag

SEQ ID NO: 71 - protein, 15 aa, Artificial Sequence
  Synthetic construct
IINTDGSAYYATWAK

SEQ ID NO: 72 - DNA, 9 bp, Artificial Sequence
  Synthetic construct
ggtagcgcg

SEQ ID NO: 73 - protein, 3 aa, Artificial Sequence
  Synthetic construct
GSA

SEQ ID NO: 74 - DNA, 18 bp, Artificial Sequence
  Synthetic construct
caggccagtcagactgtt

SEQ ID NO: 75 - protein, 6 aa, Artificial Sequence
  Synthetic construct
QASQTV

SEQ ID NO: 76 - DNA, 21 bp, Artificial Sequence
  Synthetic construct
tctgcatccactctggcatct

SEQ ID NO: 77 - protein, 7 aa, Artificial Sequence
  Synthetic construct
SASTLAS

SEQ ID NO: 78 - DNA, 39 bp, Artificial Sequence
  Synthetic construct
ctaggcgcttgggattgtcgtagtgctgattgttttgct

SEQ ID NO: 79 - protein, 13 aa, Artificial Sequence
  Synthetic construct
LGAWDCRSADCFA

(19) United States
(12) Patent Application Publication
(10) Pub. No.: US 2009/0233358 A1
(43) Pub. Date: Sep. 17, 2009

(54) Method of Regulating A Phosphorylated Protein-Mediated Intracellular Signal Transduction Using An Antibody Specifically Binding To The Phosphorylated Protein

(75) Inventors: Sue Goo Rhee, Seoul (KR); Jong Seo Lee, Seoul (KR); Junho Chung, Seongnam-si (KR); Hyunbo Shim, Seoul (KR); Bong Kook Ko, Seoul (KR); Mi Young Koo, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Young In Frontier Co., Ltd., Seoul (KR)

(21) Appl. No.: 12/393,258

(22) Filed: Feb. 26, 2009

Related U.S. Application Data
(60) Provisional application No. 61/031,433, filed on Feb. 26, 2008.

(30) Foreign Application Priority Data
Mar. 12, 2008 (KR) 10-2008-0023019

(51) Int. Cl. C12N 5/02; C07H 21/04

(52) U.S. Cl. 435/375; 536/23.53

(57) ABSTRACT

There are provided a method of regulating a phosphorylated protein-mediated intracellular signal transduction comprising intracellularly expressing an antibody that specifically binds to the phosphorylated protein and an expression system for intracellular expression of the antibody. The method and system are effectively used in the investigation, prevention, or treatment of diseases caused by a phosphorylated protein-mediated intracellular signal transduction, including prostate cancer, lung cancer and breast cancer, through regulation of a molecular interaction involving a phosphorylated residue of the phosphorylated protein.

### FIELD OF THE INVENTION

The present invention relates to a method of regulating the phosphorylated protein-mediated intracellular signal transduction, and more particularly, to a method of regulating a molecular interaction involving a phosphorylated residue of a phosphorylated protein, which comprises intracellularly expressing an antibody specifically binding to the phosphorylated protein; and an expression system for intracellular expression of the antibody.

### BACKGROUND OF THE INVENTION

Proteins are assembled based on the genetic information recorded in DNAs through transcription and translation. Most of such proteins are structural proteins that maintain cellular skeletal structures, but many proteins are active proteins that interact with other cellular products. The regulation of the activity of proteins plays a critical role in the intracellular signal transduction for regulating intracellular functions of the proteins.

The intracellular signal transduction mediated by a protein can be regulated through an on/off regulatory switch for the activation of the protein. The activation or inactivation of a protein can be achieved by various post-translational modification methods such as phosphorylation, glycosylation, methylation, acetylation and protein-protein interaction. Such post-translational modification methods play critical roles in intracellular signal transduction. Among them, protein phosphorylation is one of many mechanisms that control gene expression by signals induced by extracellular or intracellular stimuli (Manning G. et al., the protein kinase complement of the human genome, Science 298: 1912-1934 (2002)).

It is known that the phosphorylation of a protein mediates intracellular signal transduction by affecting, among other, the activity and structure of the protein as well as the binding of the protein to other protein(s) (Hunter T. et al., Tyrosine phosphorylation in cell signaling and disease, Keio J. Med, 51:61-71 (2002)). With the improvements and advances of molecular biological knowledge, new drugs designed based on protein phosphorylation have been developed. For example, there are drugs capable of inhibiting the binding activity of proteins to phosphorylated residues of phosphorylated proteins.

Stat3 (signal transducers and activators of transcription 3), which is a signal transducer inducing cell proliferation, is phosphorylated in response to an extracellular signal molecule, such as interleukin (IL) or interferon-gamma (IFN-γ), to form a homodimer or a heterodimer which is translocated into the nucleus to induce the expression of a target gene. In particular, as Stat3 is known to be oncogenic, there is an increasing interest in Stat3. Actually, abnormal activation of Stat3 in various tumor tissues has been reported, and thus, many researchers have participated in developing anticancer drugs capable of suppressing the abnormal activation of Stat3. However, the development of anticancer drugs capable of suppressing the activation of Stat3 is still at an early stage (Frank D A. Mol Med. 1999 July; 5(7): 432-456; and Buettner R. et al., Clin Cancer Res. 2002 April; 8(4): 945-954).

Phospholipase C (PLC) hydrolyzes phosphatidyl-inositol 4,5-bisphosphate (PIP2), a phospholipid present in a small amount in cell membranes, to produce diacylglycerol (DG) and inositol 1,4,5-triphosphate (IP3). DG serves as a messenger for the activation of protein kinase C (PKC) in cells and IP3 binds to an IP3 receptor of endoplasmic reticulum (ER) to induce signal transduction through intracellular Ca²⁺ release. It has been reported that when protein tyrosine kinase (PTK) is activated in response to an external stimulus such as a platelet-derived growth factor (PDGF), an epidermal growth factor (EGF) or a nerve growth factor (NGF), signal transduction is initiated through phosphorylation of the tyrosine residues of PLC-γ isozymes (Rhee S. G. et al., Regulation of phosphoinositide-specific phospholipase C isozymes. J. Biol. Chem. 272: 15045-15048, 1997; and Kamat A. et al., phospholipase C-gamma 1: Regulation of enzyme function and role in growth factor-dependent signal transduction, Cytokine Growth Factor Rev. 8: 109-117, 1997).

It has been reported that the elevation of the activity and expression level of PLC-γ induces tumorigenesis of normal cells (Peng T. et al., Cardiovasc Res. Jan. 17 (2008); and Liu J. et al., Ai Zheng, 2007 Sep. 26(9): 957-962).

Akt, also known as a protein kinase B, is an evolutionarily preserved serine/threonine-specific protein kinase and there are three mammalian isoforms: Akt1, Akt2, and Akt3 (Altomare D A et al., Oncogene 24:7455 (2005)). It has been known that Akt is activated through phosphorylation of Thr308 or Ser473, a pleckstrin homology domain of Akt, by an action of phosphatidylinositol 3 kinase (PI3K) and the activated Akt is responsible for regulating various cellular functions, including cell survival, proliferation, and other metabolic processes (Hennessy B T et al., Nat Rev Drug Discov 4:988 (2005); Powis G et al., Clin Cancer Res 12:2964-34 (2006)). That has been reported that Akt phosphorylation occurs more frequently in various cancer patients than in normal persons (James A et al., Mol Cancer Ther 6: 2139 (2007)). Thus, an attempt to suppress carcinogenesis through alteration of the Akt-mediated intracellular signal transduction has been actively pursued. Clinical trials have shown that Akt phosphorylation is used as a prognostic factor for an early stage lung cancer in humans and that Akt is activated by a cigarette smoke-associated carcinogen (West K A et al., J Clin Invest 111:80 (2003), 28-30; West K A et al., Cancer Res 64:446 (2004); Chun K H, J Natl Cancer Inst 95:291 (2003)).

A method of eliminating or significantly reducing the function of a target antigen protein by expressing an antibody which binds to the target antigen has been reported (A. S.-Y. Lo et al., Therapeutic Antibody, Handbook of Experimental Pharmacology 181). An intracellular antibody, also known as “intrabody”, has been used as a biotechnological tool for eliminating or regulating the function of a target antigen at the post-translational level. An intracellular antibody is an antibody designed to be expressed intracellularly and to specifically bind to a target antigen present in various subcellular locations including the cytosol, nucleus, and endoplasmic reticulum (ER). For example, when an antibody against a receptor protein is targeted to the endoplasmic reticulum, the receptor is expressed but is not expressed in the endoplasmic reticulum in any detectable amount. Further, the down-regulation of a transcriptional factor by targeting an antibody against the transcriptional factor to the cytosol or nucleus has been reported.

An intracellular antibody can be expressed in any one of various antibody forms, e.g., in the form of a single chain variable fragment (scFv) antibody composed of heavy- and light-chain variable regions that are joined by an interchain linker (ICL). An intracellular antibody has been used in diverse studies of cancer, HIV, or autoimmune diseases (Lo A S et al., Handb Exp Pharmacol. 181: 343-373 (2008)).

However, intracellular expression of an antibody specifically binding to a phosphorylated protein has not yet been reported.

### SUMMARY OF THE INVENTION

Accordingly, it is an object of the present invention to provide a method of regulating the molecular interaction involving a phosphorylated residue of a phosphorylated protein.

It is another object of the present invention to provide a method of treating a disease caused by the molecular interaction involving the phosphorylated residue of a phosphorylated protein.

It is still another object of the present invention to provide an expression system for intracellular expression of an antibody that specifically binds to a phosphorylated protein.

According to an aspect of the present invention, there is provided a method of regulating the phosphorylated protein-mediated intracellular signal transduction, comprising intracellularly expressing an antibody that specifically binds to the phosphorylated protein.

In an embodiment of the present invention, the antibody regulates the molecular interaction involving the phosphorylated residue of said phosphorylated protein.

In another embodiment of the present invention, the antibody stabilizes the phosphorylated protein or prolongs the survival of the phosphorylated protein.

According to another aspect of the present invention, there is provided a method of treating or preventing a disease caused by the molecular interaction involving the phosphorylated residue of a phosphorylated protein, the method comprising introducing into a target cell a vector having a polynucleotide encoding an antibody that specifically binds to the phosphorylated protein or to an antigen-binding fragment thereof.

According to still another aspect of the present invention, there is provided an expression system for intracellular expression of an antibody that specifically binds to a phosphorylated protein, which is inclusive of a nucleic acid molecule encoding the antibody.

### BRIEF DESCRIPTION OF THE DRAWINGS

The above and other objects and features of the present invention will become apparent from the following description of the invention, when taken in conjunction with the accompanying drawings, which respectively show:

FIG. 1: a schematic flow diagram illustrating a method of manufacturing an antibody that is expressed intracellularly and specifically binds to a phosphorylated protein;

FIG. 2: an immunoblot result showing that anti-pStat3 Fab antibody that specifically binds to pStat3;

FIG. 3: an immunoprecipitation result showing that anti-pStat3 scFv-Fc antibody specifically binds to pStat3;

FIG. 4A: a diagram illustrating a green fluorescent protein (GFP)-fused scFv antibody specific for pStat3 protein;

FIG. 4B: a view illustrating that anti-pStat3 scFv antibody binds to pStat3, as confirmed by immunoprecipitation assay followed by immunoblot assay;

FIG. 4C: images showing the translocation of pStat3 into the nuclei of anti-pStat3 scFv antibody-expressing or non-expressing cells in response to IL-6 or IFN-γ;

FIG. 5: an immunoblot result showing that the stability of pStat3 protein increases in cells transfected with a recombinant adenovirus that expresses scFv antibody specific for the pStat3 protein, regardless of the presence of an external signal transducer, IL-6;

FIGS. 6A and 6B: pStat3 present in cells expressing anti-pStat3 scFV antibody is stabilized through its binding with the antibody and its expression level increases markedly, regardless of the presence of IL-6 (FIG. 6B), over the control cells expressing EGFP (FIG. 6A), but the antibody-bound pStat3 protein does not form a homodimer or a heterodimer which is known to be translocated into the nucleus, to lower the expression of p21, a downstream target of pStat3;

FIG. 7A: the degree of Ca²⁺ mobilization in EGFP-expressing cells;

FIG. 7B: the degree of Ca²⁺ mobilization in anti-pPLC-γ scFv antibody-expressing cells; and

FIG. 8: an immunoblot result showing that pAkt is upregulated in cells expressing scFv antibody specific for pAkt regardless of the presence of insulin, to enhance the phosphorylation of PRAS40, a downstream target of pAkt, unlike EGFP-expressing control cells.

### DETAILED DESCRIPTION OF THE INVENTION

A method of regulating the phosphorylated protein-mediated intracellular signal transduction by manipulating a protein (downstream protein) that binds to a target phosphorylated site of the phosphorylated protein (upstream protein) will now be described more fully with respect to exemplary embodiments of the invention.

The present inventors have endeavored to develop a method of modifying a downstream intracellular signal transduction pathway of a phosphorylated protein using an antibody binding to the phosphorylated protein, and found that the intracellular expression of antibodies specifically binding to phosphorylated Stat3, PLC-γ and Akt induces the modification of intracellular signal transduction. Generally, Stat3 is phosphorylated at the tyrosine 705 residue in response to an extracellular signal, such as interleukin (IL) or interferon-gamma (IFN-γ), to form a homodimer or a heterodimer that translocates into the nucleus. The present inventors have found that intracellular expression of an antibody specifically binding to phosphorylated Stat3 (hereinafter, also referred to simply as “pStat3”) inhibits the translocation of pStat3 into the nucleus. The present inventors have also found that when cells are transfected with an adenovirus expressing an antibody specifically binding to pStat3, pStat3 is stabilized and overexpressed through binding with the antibody expressed in the cells, regardless of the presence of a signal transducer, IL, or IFN-γ and thus, the antibody-bound pStat3 does not form a homodimer or a heterodimer translocating into the nucleus, leading to the failure of activation of p21, a downstream target of pStat3. On the other hand, the present inventors have found that the intracellular expression of an antibody specifically binding to phosphorylated PLC-γ (hereinafter, also referred to simply as “pPLC-γ”) inhibits Ca²⁺ mobilization, and when the phosphorylation of Akt is induced in the presence of insulin, the amount of phosphorylated Akt (hereinafter, also referred to simply as “pAkt”) increases regardless of the presence of an extracellular signal transducer, leading to increased expression of pPRAS40 (T246), a downstream protein of the PI13K/Akt signal transduction pathway.

Therefore, the present invention provides a method of regulating a molecular interaction involving a phosphorylated residue of a phosphorylated protein, comprising intracellularly expressing an antibody that specifically binds to the phosphorylated protein.

The phosphorylated residue of the phosphorylated protein may be tyrosine or serine.

The phosphorylated protein may be pStat3, pPLC-γ, pAkt, or others.

The antibody may include heavy-chain complementarity determining regions (CDRs) of SEQ ID NOS: 8, 10 and 12, and light-chain CDRs of SEQ ID NOS: 14, 16 and 18. Alternatively, the antibody may include heavy-chain CDRs of SEQ ID NOS: 26, 28 and 30 and light-chain CDRs of SEQ ID NOS: 32, 34 and 36, or heavy-chain CDRs of SEQ ID NOS: 69, 71 and 73 and light-chain CDRs of SEQ ID NOS: 75, 77 and 79. The antibody may be expressed in various antibody forms, generally in the form of a single chain variable fragment (scFv) antibody composed of heavy- and light-chain variable regions that are joined by an interchain linker (ICL). More preferably, the antibody may be antibody scFv having the amino acid sequence of SEQ ID NO: 2, 20 or 63.

The inventive method may comprise introducing into a cell, a vector having a polynucleotide encoding the antibody or an antigen-binding fragment thereof.

The polynucleotide may include heavy-chain CDR-encoding nucleotide sequences of SEQ ID NOS: 7, 9 and 11, and light-chain CDR-encoding nucleotide sequences of SEQ ID NOS: 13, 15 and 17, a nucleotide sequence of SEQ ID NO: 1 being more preferred. Alternatively, the polynucleotide may include heavy-chain CDR-encoding nucleotide sequences of SEQ ID NOS: 25, 27 and 29, and light-chain CDR-encoding nucleotide sequences of SEQ ID NOS: 31, 33 and 35, a nucleotide sequence of SEQ ID NO: 19 being more preferred. Alternatively, the polynucleotide may include heavy-chain CDR-encoding nucleotide sequences of SEQ ID NOS: 68, 70 and 72, and light-chain CDR-encoding nucleotide sequences of SEQ ID NOS: 74, 76 and 78, a nucleotide sequence of SEQ ID NO: 62 being more preferred.

The vector may be optionally selected from promoter-containing vectors that can express a protein in a mammalian cell, e.g., a commercially available plasmid vector, pEGFPC1 (Clontech).

According to the inventive method, the antibody specifically binding to the phosphorylated protein can be expressed in target cells used for the investigation, prevention or treatment of diseases involving a phosphorylated protein-mediated intracellular signal transduction.

Thus, the present invention also provides a method of treating or preventing diseases caused by a molecular interaction involving a phosphorylated residue of a phosphorylated protein, comprising introducing into a cell a vector having a polynucleotide encoding an antibody that specifically binds to the phosphorylated protein or an antigen-binding fragment thereof. Specifically, the diseases caused by the molecular interaction involving the phosphorylated residue of the phosphorylated protein may be various cancers including prostate cancer, lung cancer and breast cancer, and immune diseases, or others.

The introduction of the inventive vector into the cell may be performed by a method commonly known in the art to introduce a foreign gene-containing vector into a cell. For example, established cancer cell lines, e.g., HeLa cervical cancer cell line, may be transfected with a plasmid vector constructed by inserting into a plasmid a nucleotide sequence encoding an antibody that specifically binds to pStat3.

The present invention also provides an expression system for intracellular expression of an antibody specifically binding to a phosphorylated protein, which is inclusive of a nucleic acid molecule encoding the antibody.

The nucleic acid molecule may include heavy-chain CDR-encoding nucleotide sequences of SEQ ID NOS: 7, 9 and 11, and light-chain CDR-encoding nucleotide sequences of SEQ ID NOS: 13, 15 and 17, a nucleotide sequence of SEQ ID NO: 1 being more preferred. Alternatively, the nucleic acid molecule may include heavy-chain CDR-encoding nucleotide sequences of SEQ ID NOS: 25, 27 and 29, and light-chain CDR-encoding nucleotide sequences of SEQ ID NOS: 31, 33 and 35, a nucleotide sequence of SEQ ID NO: 19 being more preferred. Alternatively, the nucleic acid molecule may include heavy-chain CDR-encoding nucleotide sequences of SEQ ID NOS: 68, 70 and 72, and light-chain CDR-encoding nucleotide sequences of SEQ ID NOS: 74, 76 and 78, a nucleotide sequence of SEQ ID NO: 62 being more preferred.

The expression system may be an expression tool such as an expression kit. For example, the expression kit may be a kit designed specifically to highly express the antibody specifically binding to the phosphorylated protein in a target cell used for the investigation, prevention and treatment of diseases involving a phosphorylated protein-mediated intracellular signal transduction, and may include a vector for intracellular expression of the antibody, a transfection reagent, a user manual, or others.

The present invention also provides a method of regulating a phosphorylated protein-mediated intracellular signal transduction by stabilizing or sustaining the phosphorylation of a phosphorylated residue of the phosphorylated protein, comprising intracellularly expressing an antibody that specifically binds to the phosphorylated protein.

The phosphorylated residue of the phosphorylated protein may be tyrosine or serine.

The phosphorylated protein may be pStat3, pPLC-γ, pAkt, or others.

The antibody may include heavy-chain CDRs of SEQ ID NOS: 8, 10 and 12, and light-chain CDRs of SEQ ID NOS: 14, 16 and 18. Alternatively, the antibody may include heavy-chain CDRs of SEQ ID NOS: 26, 28 and 30 and light-chain CDRs of SEQ ID NOS: 32, 34 and 36; or heavy-chain CDRs of SEQ ID NOS: 69, 71 and 73 and light-chain CDRs of SEQ ID NOS: 75, 77 and 79. The antibody may be expressed in various antibody forms, generally in the form of antibody scFv composed of heavy- and light-chain variable regions that are joined by an ICL. More preferably, the antibody may be antibody scFv having an amino acid sequence of SEQ ID NO: 2, 20 or 63.

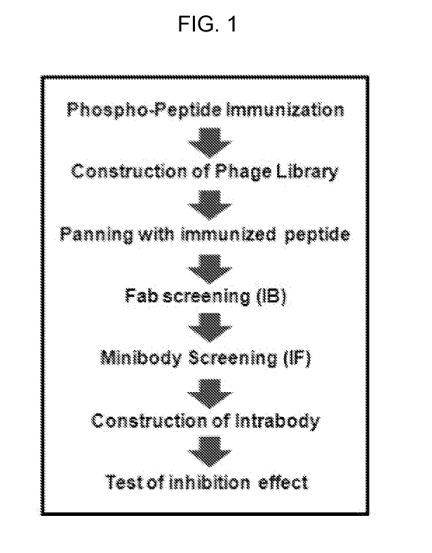

FIG. 1 schematically illustrates a method of manufacturing an antibody that is expressed intracellularly and specifically binds to a phosphorylated protein. The present invention will be described in more detail by way of the following examples with reference to FIG. 1. However, the following examples are only for illustrative purposes and are not intended to limit the scope of the invention.

### Example 1

### Phospho-Peptide Immunization and Construction of an Antibody Phage Library

In order to immunize rabbits with phospho-peptides, the phospho-peptides of Stat3, PLC-γ and Akt, i.e., pStat3 (PGSAAP-pY-LKTKGGGSC (SEQ ID NO: 59); pStat3 (Y705)), pPLC-γ (RNPGF-pY-VEANPGGGSC (SEQ ID NO: 60); pPLC-γ (Y783)) and pAkt (PHFPQF-pS-YSASGGGSC (SEQ ID NO: 61); pAkt (S473)) were first synthesized by post-translational modification (Thermo Scientific).

The phospho-peptides thus synthesized were conjugated with an immunogenic carrier protein, KLH (keyhole limpet hemocyanin, Pierce) or OVA (ovalbumin, Sigma) and rabbits (New Zealand White, 2.5 kg) were then immunized with the peptide-KLH/OVA conjugates. At this time, in order to reduce the possibility of producing an antibody against the carrier protein, the immunization was induced through alternate use of KLH and OVA. Blood samples were taken from the immunized rabbits, and ELISA was performed using the peptide used as the immunogen to determine whether or not antibodies against pStat3 (Y705), pPLC-γ (Y783) and pAkt (S473) were detected.

The construction of an antibody phage library was performed as follows according to a Barbas's document (see Barbas et al., Phage Display: A laboratory manual, Cold Spring Harbor Laboratory Press, Section 2, Chapter 8-9 (2001)). Total RNA was extracted from the spleen and bone marrow of rabbits identified to produce the antibodies, and RT-PCR was performed using a Superscript III First-Strand Synthesis System (Invitrogen) to synthesize cDNAs.

PCR was performed using cDNAs as templates and primer sequences presented in Tables 1 and 2 below. As a result, genes encoding heavy- and light-chain variable regions of antibodies against pStat3, pPLC-γ and pAkt were obtained.

TABLE 1
Primers for heavy-chain variable regions of antibodies against pStat3, pPLC-γ and pAkt

| | |
|---|---|
| Primer | Sequence |

| | |
|---|---|
| Sense | 5′-GCT GCC CAA CCA GCC ATG GCC CAG TCG GTG GAG GAG TCC RGG-3′ (SEQ ID NO: 37) |
| | 5′-GCT GCC CAA CCA GCC ATG GCC CAG TCG GTG AAG GAG TCC GAG-3′ (SEQ ID NO: 38) |
| | 5′-GCT GCC CAA CCA GCC ATG CGG CAG TCG YTG GAG GAG TCC GGG-3′ (SEQ ID NO: 39) |
| | 5′-GCT GCC CAA CCA GCC ATG GCC CAG SAG CAG CTG RTG GAG TCC GG-3′ (SEQ ID NO: 40) |
| Antisense | 5′-CGA TGG GCC CTT GGT GGA GGC TGA RGA GAY GGT GAC CAG GGT GCC-3′ (SEQ ID NO: 41) |

TABLE 2
Primers for light-chain variable regions of antibodies against pStat3, pPLC-γ and pAkt

| | |
|---|---|
| Primer | Sequence |

| | |
|---|---|
| Sense | 5′-GGG CCC AGG CGG CCG AGC TCG TGM TGA CCC AGA CTC CA-3′ (SEQ ID NO: 42) |
| | 5′-GGG CCC AGG CGG CCG AGC TCG ATM TGA CCC AGA CTC CA-3′ (SEQ ID NO: 43) |
| | 5′-GGG CCC AGG CGG CCG AGC TCG TGA TGA CCC AGA CTG AA-3′ (SEQ ID NO: 44) |
| | 5′-GGG CCC AGG CGG CCG AGC TCG TGC TGA CTC AGT CGC CCT C-3′ (SEQ ID NO: 45) |
| Antisense | 5′-AGA TGG TGC AGC CAC AGT TCG TTT GAT TTC CAC ATT GGT GCC-3′ (SEQ ID NO: 46) |
| | 5′-AGA TGG TGC AGC CAC AGT TCG TAG GAT CTC CAG CTC GGT CCC-3′ (SEQ ID NO: 47) |
| | 5′-AGA TGG TGC AGC CAC AGT TCG TTT GAC SAC CAC CTC GGT CCC-3′ (SEQ ID NO: 48) |
| | 5′-AGA TGG TGC ACG CAC AGT TCG GCC TGT GAC GGT CAG CTG GGT CCC-3′ (SEQ ID NO: 49) |

Meanwhile, PCR was performed using the pComb3XTT vectors (Barbas laboratory) as templates and primer sequences presented in Table 3 below. As a result, genes encoding human heavy-chain constant regions (CH1) and human light-chain constant regions (Cκ) were obtained.

TABLE 3
Primers for human CH1 and Cκ

| | |
|---|---|
| Primer | Sequence |

| | | |
|---|---|---|
| Human | Sense | 5′-GCC TCC ACC AAG GGC CCA TCG GTC-3′ (SEQ ID NO: 50) |
| CH1 | Antisense | 5′-AGA AGC CTA GTC CGG AAC GTC-3′ (SEQ ID NO: 51) |
| Human | Sense | 5′-CGA ACT GTG GCT GCA CCA TCT GTC-3′ (SEQ ID NO: 52) |
| Cκ | Antisense | 5′-GGC CAT GGC TGG TTG GGC AGC-3′ (SEQ ID NO: 53) |

Genes encoding Fab (antigen-binding fragment) antibodies were obtained from the above-synthesized heavy- and light-chain variable and constant regions of the antibodies against pStat3, pPLC-γ and pAkt through overlap PCR using primers presented in Tables 4 and 5 below.

TABLE 4

Primers for sequential amplification of each
heavy-chain variable region of the antibodies
against pStat3, pPLC-γ and pAkt, and human CH1

| | |
|---|---|
| Primer | Sequence |

| | |
|---|---|
| Sense | 5′-GCT GCC CAA CCA GCC ATG GCC-3′ (SEQ ID NO: 54) |
| Antisense | 5′-AGA AGC GTA GTC CGG AAC GTC-3′ (SEQ ID NO: 51) |

TABLE 5
Primers for sequential amplification of each light-chain variable
region of the antibodies against pStat3, pPLC-γ and pAkt, and human Cκ

| | |
|---|---|
| Primer | Sequence |

| | |
|---|---|
| Sense | 5′-GAG GAG GAG GAG GAG GAG GCG GGG CCC AGG CGG CCG AGC TC-3′ |
| | (SEQ ID NO: 55) |
| Antisense | 5′-GGC CAT GGC TGG TTG GGC AGC-3′ (SEQ ID NO: 53) |

Then, overlap PCR was performed using the above-obtained heavy- and light-chain encoding genes and primers presented in Table 6 below to obtain PCR products (light-chain variable region+human Cκ+heavy-chain variable region+human CH1), and the PCR products were then inserted into pComb3XSS vectors (Barbas laboratory) to thereby construct an antibody plasmid library.

TABLE 6

Primers for sequential amplification of each light-chain variable region of the
antibodies against pStat3, pPLC-γ and pAkt, human Cκ, each heavy-chain variable
region of the antibodies against pStat3, pPLC-γ and pAkt, and human CH1

| | |
|---|---|
| Primer | Sequence |

| | |
|---|---|
| Sense | 5′-GAG GAG GAG GAG GAG GAG GCG GGG CCC AGG CGG CCG AGC TC-3′ (SEQ ID NO: 55) |
| Antisense | 5′-GAG GAG GAG GAG GAG GAG AGA AGC GTA GTC CGG AAC GTC-3′ (SEQ ID NO: 56) |

The antibody plasmid library was transformed into ER2537 bacteria (New England Biolabs) and then into VCSM13 helper phages (Stratagene) to construct an antibody phage library.

### Example 2

### Screening of Fab Clones

The pStat3, pPLC-γ and pAkt peptides used as immunogens in Example 1 were bound to bovine serum albumin (BSA, Sigma) and then to magnetic beads (Dynabead M-270 Epoxy, Invitrogen) to obtain peptide-BSA-beads. The thus-obtained peptide-BSA-beads and the phage library obtained in Example 1 were incubated in a TBS-T-BSA buffer (50 mM Tris, 150 mM NaCl, 0.05% Triton X-100, 5% BSA, pH 7.4) at room temperature for one hour. The obtained resultants were washed with a TBS-T buffer (50 mM Tris, 150 mM NaCl, 0.05% Triton X-100, pH 7.4) once for 10 minutes, five times for 10 minutes, and 10 times for 10 minutes to remove nonspecific phages and obtain phages specifically bound to the beads with varying pH.

As a result, a heavy-chain variable region of a Fab antibody against pStat3 had a nucleotide sequence of SEQ ID NO: 3 and an amino acid sequence of SEQ ID NO: 4, and a light-chain variable region of the Fab antibody against pStat3 had a nucleotide sequence of SEQ ID NO: 5 and an amino acid sequence of SEQ ID NO: 6. A heavy-chain variable region of a Fab antibody against pPLC-γhad a nucleotide sequence of SEQ ID NO: 21 and an amino acid sequence of SEQ ID NO: 22, and a light-chain variable region of the Fab antibody against pPLC-γ had a nucleotide sequence of SEQ ID NO: 23 and an amino acid sequence of SEQ ID NO: 24. A heavy-chain variable region of a Fab antibody against pAkt had a nucleotide sequence of SEQ ID NO: 64 and an amino acid sequence of SEQ ID NO: 65, and a light-chain variable region of the Fab antibody against pAkt had a nucleotide sequence of SEQ ID NO: 66 and an amino acid sequence of SEQ ID NO: 67. The Fab antibodies were used after purification on a Ni-NTA resin (Qiagen) binding with a COOH-terminal His tag.

### Example 3

### Immunoblot (IB) Assay for Detecting the Binding of Phosphorylated Proteins and their Fab Antibodies in Cells

First, in order to obtain cells containing phosphorylated Stat3 (pStat3), HeLa cells (American Type Culture Collection (ATCC)) were cultured in a fetal bovine serum (FBS)-free DMEM (High glucose, HyClone) for 24 hours and treated with IFN-α (150 ng/ml) for 15 minutes. In order to obtain cells containing phosphorylated PLC-γ (pPLC-γ) or Akt (pAkt), NIH3T3 cells (ATCC) were cultured in a fetal calf serum (FCS)-free DMEM (High glucose, HyClone) for four hours and treated with PDGF (100 ng/ml) for 20 minutes.

Cells extracts were obtained from the HeLa and NIH3T3 cell lines thus prepared as follows. First, the cells were washed twice with PBS (137 mM NaCl, 2.7 mM KCl, 12 mM Na₂HPO₄, 1.2 mM KH₂PO₄, pH 7.4), and an IB buffer (20 mM HEPES, pH 7.2; 20 mM phosphoglycerate; 150 mM NaCl; 10% glycerol; 1% NP-40; 1 mM EDTA; 1 mM EGTA; 1 mM PMSF, 1 uM Leupeptin, 0.3 uM Aprotinin, 0.3 mM Pepstatin) was added thereto. Then, the cells were incubated at 4° C. for 30 minutes and centrifuged at 15,000 rpm for 15 minutes to obtain cell extracts (supernatants).

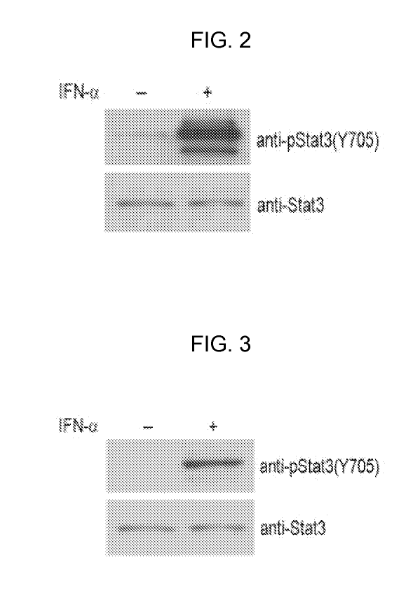

An immunoblot assay was performed with the cell extracts, the Fab antibodies against pStat3, pPLC-γ and pAkt obtained in Example 2 as primary antibodies, and a HRP conjugated anti-HA antibody (Roche) as a secondary antibody. The immunoblot assay result for the anti-pStat3 Fab antibody is shown in FIG. 2.

As shown in FIG. 2, as reported previously, a predominant band corresponding to the size of a pStat3 protein was detected in the cells treated with IFN-α, unlike the cells untreated with IFN-α. This result suggests that the anti-pStat3 antibody obtained in Example 2 specifically binds to pStat3. The immunoblot results also showed that the anti-pPLC-γ and pAkt antibodies were specifically bound to pPLC-γ and pAkt proteins, respectively.

### Example 4

### Construction of scFv-Fc Minibody

In order to obtain scFv-Fc minibodies specific for pStat3, pPLC-γ and pAkt, the heavy- and light-chain variable regions of the Fab antibodies obtained in Example 2 were linked by an amino acid linker (5′-GGSSRSSSSGGGGSGGGG-3′; SEQ ID NO: 58) to obtain single-chain variable fragment (scFv) antibodies. Six CDRs of scFv against pStat3 had nucleotide sequences of SEQ ID NOS: 7, 9, 11, 13, 15 and 17, and amino acid sequences of SEQ ID NOS: 8, 10, 12, 14, 16 and 18. Six CDRs of scFv against pPLC-γ had nucleotide sequences of SEQ ID NOS: 25, 27, 29, 31, 33 and 35, and amino acid sequences of SEQ ID NOS: 26, 28, 30, 32, 34 and 36. Six CDRs of scFv against pAkt had nucleotide sequences of SEQ ID NOS: 68, 70, 72, 74, 76 and 78, and amino acid sequences of SEQ ID NOS: 69, 71, 73, 75, 77 and 79. The scFv antibodies were bound to the Fc region of a linker-fused human IgG1, cloned into pcDNA3.1 vectors (Invitrogen), and transiently transfected into 293F cells (Invitrogen) to construct scFv-Fc minibodies. The minibodies were used after purification using protein-G beads (Amersham).

### Example 5

### Immunoprecipitation (IP) Assay for Specificities of scFv-Fc Minibodies

In this Example, the specificities of the scFv-Fc antibodies obtained in Example 4 were analyzed using an immunoprecipitation (IP) assay.

For this, IFN-α-treated or untreated HeLa cells or PDGF-treated or untreated NIH3T3 cells were treated with an IP buffer (50 mM Tris-HCL, pH 7.4; 150 mM NaCl, 1% NP-40, 1 mM EDTA, 1 mM PMSF, 1 uM Leupeptin, 0.3 uM Aprotinin, 0.3 mM Pepstatin) to obtain cell lysates. The cell lysates were immunoprecipitated with an equal amount of each of the scFv-Fc antibodies against pStat3, pPLC-γ and pAkt obtained in Example 4.

The IP result for the specificity of the scFv-Fc antibody against pStat3 is shown in FIG. 3. As shown in FIG. 3, the IP assay showed that more pStat3 proteins were expressed in the IFN-α-treated cell lysate, than in the IFN-α-untreated cell lysate. This result suggests that the cell lysates were normally prepared, and the anti-pStat3 antibody obtained in Example 4 were specifically bound to a pStat3 protein. The IP assay also showed that the anti-pPLC-γ and pAkt antibodies obtained in Example 4 were specifically bound to pPLC-γ and pAkt proteins, respectively.

### Example 6

### Evaluation for Inhibition of Nuclear Translocation of pStat3 through Intracellular Expression of an Anti-pStat3 Antibody

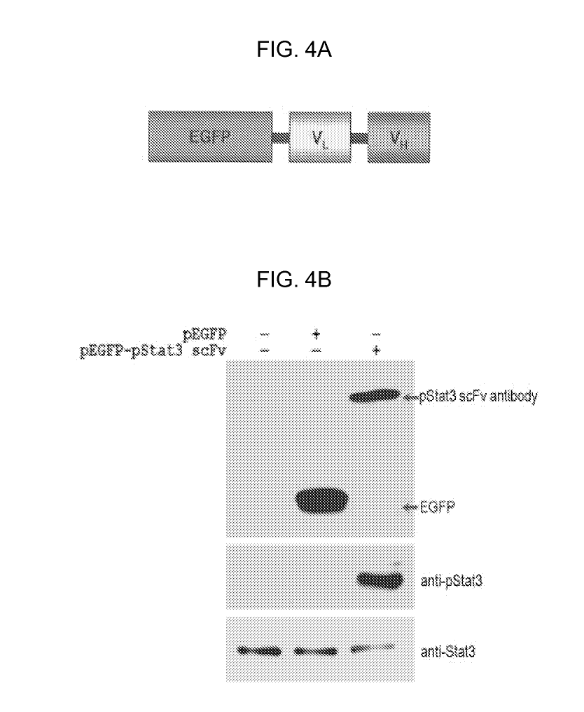

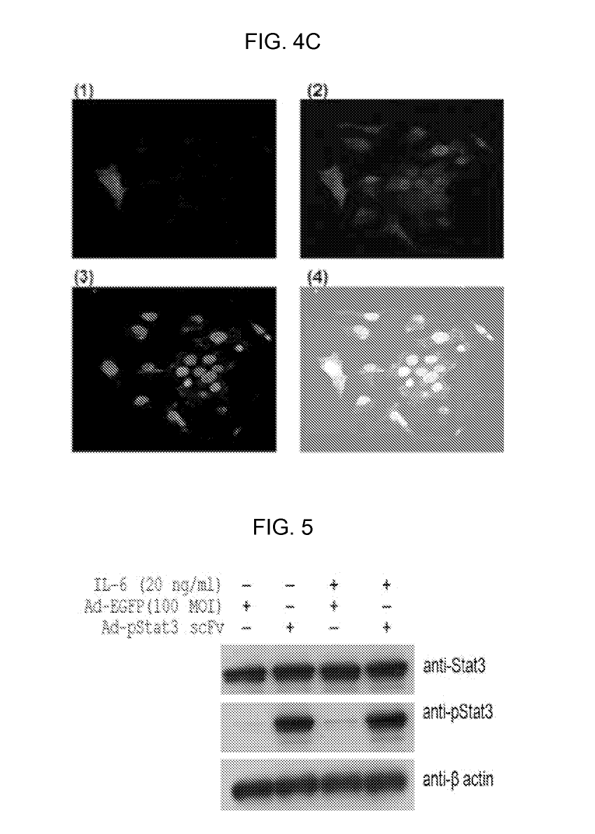

In order to investigate whether or not phosphorylated protein-mediated intracellular signal transduction is inhibited by intracellular expression of an antibody specific for the phosphorylated protein, first, a scFv antibody against pStat3 was prepared in the form of a green fluorescent protein (GFP)-fusion protein. In detail, the anti-pStat3 scFv antibody obtained in Example 4 was cloned into XhoI/HindIII restriction sites of a pEGFP-C1 vector (Clontech), and the presence of a desired DNA was identified by DNA sequencing. The nucleotide sequence and amino acid sequence of the anti-pStat3 scFv antibody were respectively represented by SEQ ID NOS: 1 and 2. A schematic structure of the scFv antibody is shown in FIG. 4A. Then, the anti-pStat3 scFv-containing vector was transfected into HeLa cells.

In order to investigate whether or not the anti-pStat3 antibody is normally expressed in the HeLa cells and binds to a pStat3 protein, IP assay was performed using an anti-GFP antibody (AbFrontier, Korea).

As shown in FIG. 4B, IP assay showed that a pStat3 protein was detected only in the antibody-expressing cells. This result suggests that the anti-pStat3 scFv antibody was expressed in the HeLa cells and bound to a pStat3 protein.

In order to investigate whether or not pStat3-mediated intracellular signal transduction is inhibited through intracellular expression of an anti-pStat3 scFv antibody, the above-prepared GFP-fused anti-pStat3 scFv antibody-encoding DNA was transfected into HepG2 cells (ATCC). After 24 hours, the cells were cultured in a FBS-free DMEM (High glucose, HyClone) for 12 hours and then treated with IL-6 (50 ng/ml) and IFN-γ (100 ng/ml) for 40 minutes. Then, the cells were fixed with paraformaldehyde and immunostained with an anti-Stat3 antibody (Cell Signaling) and a Rhodamine-conjugated anti-mouse antibody (Jackson ImmunoResearch Laboratories, Inc.). Nuclear positioning was performed by DAPI (4′-6-Diamidino-2-phenylindole; Invitrogen) staining, and the DAPI-stained cells were examined with a confocal fluorescent microscope.

The results are shown in FIG. 4C. In FIG. 4C, (1), (2), (3) and (4) are images of the same cells, specifically, (1) is an image showing an antibody-expressing cell (GFP (green) staining) and an antibody non-expressing cell, (2) is an image showing a pStat3 protein (Rhodamine (red) staining) present in cells, (3) is an image showing nuclei (DAPI (blue) staining) present in cells, and (4) is a merged image of GFP (green), Rhodamine (red) and DAPI (blue) staining. As shown in FIG. 4C, in the antibody non-expressing cells (unstained cells in (1) of FIG. 4C), pStat3 was translocated into cell nuclei in response to IL-6 and IFN-γ. On the other hand, in the antibody-expressing cells (stained cells in (1) of FIG. 4C), the translocation of pStat3 into cell nuclei was inhibited. These results reveal that the function of pStat3 is inhibited by intracellular expression of an anti-pStat3 antibody.

### Example 7

### Evaluation for an Effect of Intracellular Expression of an Anti-pStat3 scFv Antibody on pStat3 Stability (in an Adenovirus Model)

In order to investigate whether or not phosphorylated protein-mediated intracellular signal transduction is inhibited by intracellular expression of an antibody specific for the phosphorylated protein, a GFP-encoding pEGFP-C1 vector (Clontech) or the GFP-fused anti-pStat3 scFv antibody-encoding DNA prepared in Example 6 was cloned into XhoI/HindIII restriction sites of a pShuttle-CMV vector (Stratagene, 240007), and the presence of a desired DNA was identified by DNA sequencing. The resultant constructs were cleaved with PmeI, and co-transfected into BJ5183 cells (Stratagene, 200154), together with a viral vector (pAdeasy-1 vector, Stratagene 240005). The resultant recombinants were selected with kanamycin. Then, the viral gene was amplified in a small scale, and the recombinant adenovirus was selected using a PacI restriction enzyme based on a plasmid size. The recombinant adenoviral DNA was digested with a PacI restriction enzyme and transfected into QBI-293A cells (Qbiogene, AES0503) using an Effectene transfection reagent (Qiagen 301425) to obtain plaques. Then, the adenoviral gene was amplified and the expression of EGFP or the anti-pStat3 antibody was monitored by observation of GFP fluorescence. Then, the recombinant adenovirus was purified by a CsCl method and quantified (LF-RK0001, Abfrontier). The thus-prepared EGFP- or pStat3-expressing adenovirus (final 100 MOI) was transfected into HepG2 cells (ATCC). After 24 hours, the cells were cultured in a FBS-free DMEM (High glucose, HyClone) for 32 hours, and treated with IL-6 (20 ng/ml) for 15 hours, and cell extracts (supernatants) were then obtained in the same manner as described in the second paragraph of Example 3.

An immunoblot assay was performed with the cell extracts, anti-Stat3 (Cell Signaling Technology, CST) and anti-pStat3 (Abcam) antibodies as primary antibodies, and a HRP conjugated anti-rabbit antibody (PIERCE) as a secondary antibody. At this time, immunoblot using an anti-actin antibody (CST) as a loading control was also performed. In the cells transfected with an anti-pStat3 scFv antibody-encoding virus, pStat3 (Y703) was stabilized and quantitatively increased in the cells, regardless of the addition of an external signal transducer, IL-6, unlike EGFP only-expressing control cells (see FIG. 5).

### Example 8

### Evaluation for Inhibition of Expression of p21, a Downstream Protein of pStat3, Through Intracellular Expression of a scFv Antibody Specific for pStat3

In order to evaluate an effect of the expression of antibody scFv specific for pStat3 on pStat3-mediated intracellular signal transduction, the GFP-fused anti-pStat3 scFv antibody-encoding DNA prepared in Example 6 was transfected into A549 cells (ATCC). As a control, a GFP only-encoding DNA was transfected into the same cells. After 24 hours, the cells were cultured in a FBS-free DMEM (High glucose, HyClone) for 12 hours and treated with IL-6 (20 ng/ml) for 30 minutes and 15 hours, and cell extracts (supernatants) were then obtained in the same manner as described in the second paragraph of Example 3.

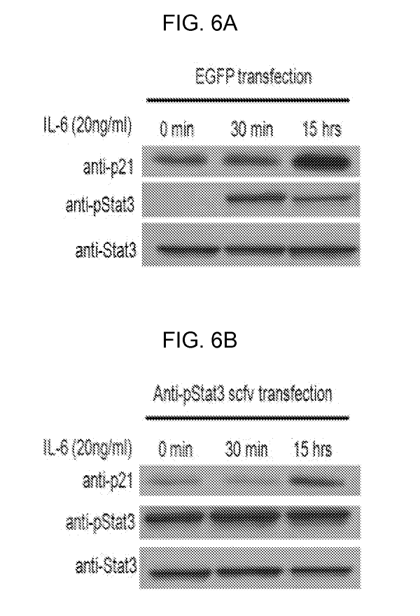

An immunoblot assay was performed with the cell extracts, anti-Stat3 (Cell Signaling Technology, CST), anti-pStat3 (Abcam), and anti-p21 (CST) antibodies as primary antibodies, and a HRP conjugated anti-rabbit antibody (PIERCE) as a secondary antibody. In the cells transfected with the anti-pStat3 scFv antibody-encoding DNA (see FIG. 6B), the expression level of pStat3 was increased regardless of the presence of an external transducer, IL-6, unlike the control cells transfected with EGFP only (see FIG. 6A). This result suggests that the stability of pStat3 was increased through binding of pStat3 to an anti-pStat3 scFv antibody expressed in cells, similarly to the results obtained in the adenovirus-transfected cells (Example 7). Even though the expression level of pStat3 was increased in the cells transfected with the anti-pStat3 scFv antibody-encoding DNA, the expression of p21, a downstream protein of pStat3, was retarded or inhibited, as compared with the EGFP only-transfected control cells. This may be because the antibody-bound pStat3 did not form a homodimer or a heterodimer translocating into the nucleus (see FIG. 4C), leading to the inactivation of p21.

### Example 9

### Evaluation for Inhibition of Ca²⁺ Mobilization Through Intracellular Expression of a scFv Antibody Specific for pPLC-γ

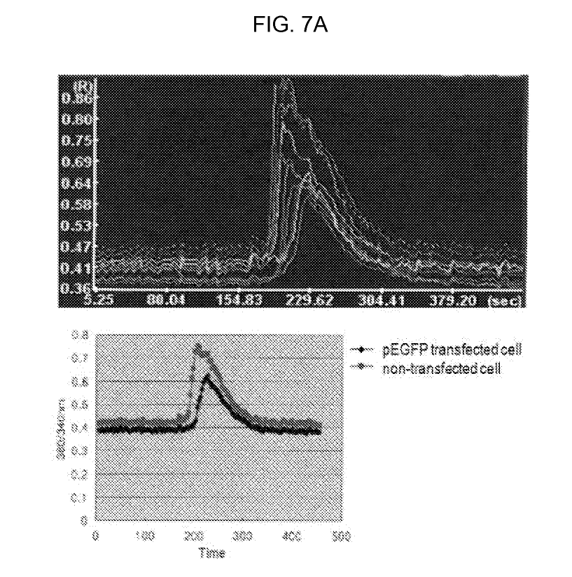

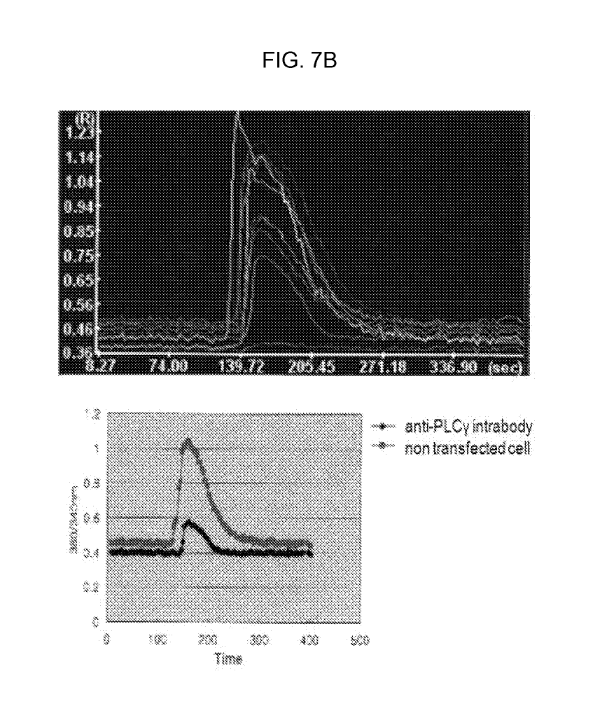

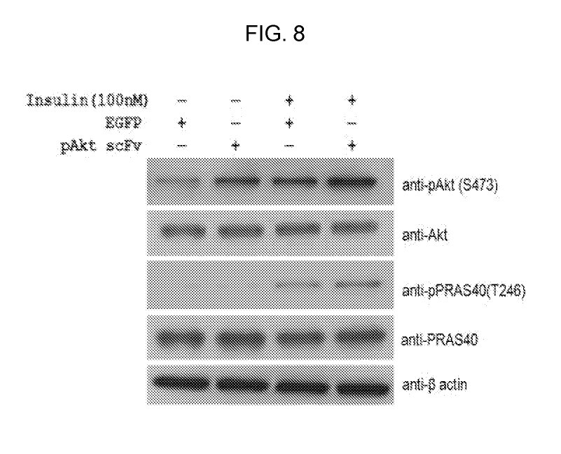

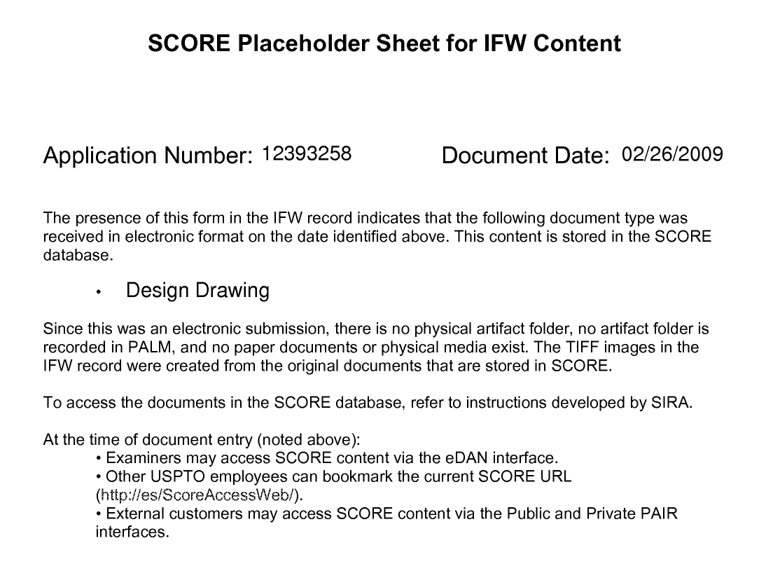

In order to evaluate an effect of an anti-pPLC-γ scFv antibody on Ca²⁺ mobilization, an anti-pPLC-γ scFv antibody was prepared in the form of a GFP-fused protein, as described in Example 6. The nucleotide sequence and amino acid sequence of the anti-pPLC-γ scFv antibody (hereinafter, referred to as “anti-pPLC-γ intrabody”) were respectively represented by SEQ ID NOS: 19 and 20. NIH3T3 cells were transfected with an anti-pPLC-γ intrabody-encoding DNA and cultured at 37° C. under a 5% CO₂ condition for 24 hours. The cells were further cultured in a FCS-free DMEM (High glucose, HyClone) for four hours, and treated with Fura-2 (5 uM; Molecular Probe) for one hour and then with PDGF (500 ng/ml) for 30 seconds in the absence of Ca²⁺. As a control, the same experiment was repeated using a GFP-encoding pEGFP-C1 vector (Clontech). Intracellular Ca²⁺ mobilization was observed with a fluorescence microscope, and the results are shown in FIGS. 7A and 7B.

As shown in FIGS. 7A and 7B, slight Ca²⁺ mobilization was observed in the EGFP-expressing cells, but Ca²⁺ mobilization was significantly reduced in the anti-pPLC-γ intrabody-expressing cells. These results reveal that pPLC-γ-mediated signal transduction is inhibited by intracellular expression of a scFv antibody specific for the pPLC-γ protein.

### Example 10

### Evaluation for the Expression Level of pPRAS40, a Downstream Protein of pAkt, Through Intracellular Expression of a scFv Antibody Specific for pAkt

In order to investigate whether or not pAkt-mediated signal transduction is inhibited by intracellular expression of an anti-pAkt scFv antibody, a GFP-fused anti-pAkt scFv antibody-encoding DNA prepared in the same manner as in Example 4 was transfected into 293T cells (ATCC). As a control, a GFP only-encoding DNA was transfected into the same cells. After 24 hours, the cells were cultured in a FBS-free DMEM (High glucose, HyClone) for 24 hours and treated with insulin (100 nM) for 20 minutes, and cell extracts (supernatants) were then obtained in the same manner as described in the second paragraph of Example 3.

An immunoblot assay was performed with the cell extracts, anti-Akt (Abfrontier), anti-pAkt (S473, SCT), and anti-pPRAS40 (T246, CST) antibodies as primary antibodies, and a HRP conjugated anti-rabbit antibody (PIERCE) as a secondary antibody. At this time, immunoblot using an anti-actin primary antibody (CST) as a loading control was also performed. In the cells transfected with the anti-pAkt scFv antibody-encoding DNA, the pAkt was increased regardless of the presence of an external signal transducer, insulin, unlike the control cells expressing EGFP only (see FIG. 8). This result reveals that when a scFv antibody specific for pAkt is expressed in cells, a target protein, pAkt is stabilized and its survival rate is prolonged in the cells, leading to increased phosphorylation of PRAS40 (T246), a downstream target of pAkt, similarly to the results obtained for pStat3 (Examples 7 and 8).

While the invention has been described with respect to the above specific embodiments, it should be recognized that various modifications and changes may be made and also fall within the scope of the invention as defined by the claims that follow.

What is claimed is:

1. A method of regulating a phosphorylated protein-mediated intracellular signal transduction, comprising intracellularly expressing an antibody that specifically binds to the phosphorylated protein.

2. The method of claim 1, wherein the antibody regulates the molecular interaction involving a phosphorylated residue of the phosphorylated protein.

3. The method of claim 2, wherein the phosphorylated reside of the phosphorylated protein is tyrosine or serine.

4. The method of claim 2, wherein the phosphorylated protein is phosphorylated Stat3, phosphorylated PLC-γ, or phosphorylated Akt.

5. The method of claim 2, wherein the antibody comprises heavy-chain complementarity determining regions (CDRs) having homologies of 70% or more with the respective amino acid sequences of SEQ ID NOS: 8, 10 and 12, and light-chain CDRs having homologies of 70% or more with the respective amino acid sequences of SEQ ID NOS: 14, 16 and 18.

6. The method of claim 2, wherein the antibody comprises heavy-chain CDRs of SEQ ID NOS: 8, 10 and 12, and light-chain CDRs of SEQ ID NOS: 14, 16 and 18.

7. The method of claim 6, wherein the antibody is antibody scFv having the amino acid sequence of SEQ ID NO: 2.

8. The method of claim 2, wherein the antibody comprises heavy-chain CDRs having homologies of 70% or more with the respective amino acid sequences of SEQ ID NOS: 26, 28 and 30, and light-chain CDRs having homologies of 70% or more with the respective amino acid sequences of SEQ ID NOS: 32, 34 and 36.

9. The method of claim 2, wherein the antibody comprises heavy-chain CDRs of SEQ ID NOS: 26, 28 and 30, and light-chain CDRs of SEQ ID NOS: 32, 34 and 36.

10. The method of claim 9, wherein the antibody is antibody scFv having the amino acid sequence of SEQ ID NO: 20.

11. The method of claim 2, wherein the antibody comprises heavy-chain CDRs having homologies of 70% or more with the respective amino acid sequences of SEQ ID NOS: 69, 71 and 73, and light-chain CDRs having homologies of 70% or more with the respective amino acid sequences of SEQ ID NOS: 75, 77 and 79.

12. The method of claim 2, wherein the antibody comprises heavy-chain CDRs of SEQ ID NOS: 69, 71 and 73, and light-chain CDRs of SEQ ID NOS: 75, 77 and 79.

13. The method of claim 12, wherein the antibody is antibody scFv having the amino acid sequence of SEQ ID NO: 63.

14. The method of claim 1, wherein the antibody stabilizes the phosphorylated protein or prolongs the survival rate of the phosphorylated protein.

15. The method of claim 14, wherein the phosphorylated reside of the phosphorylated protein is tyrosine or serine.

16. The method of claim 14, wherein the phosphorylated protein is phosphorylated Stat3 or phosphorylated Akt.

17. The method of claim 14, wherein the antibody comprises heavy-chain CDRs having homologies of 70% or more with the respective amino acid sequences of SEQ ID NOS: 8, 10 and 12, and light-chain CDRs having homologies of 70% or more with the respective amino acid sequences of SEQ ID NOS: 14, 16 and 18.

18. The method of claim 14, wherein the antibody comprises heavy-chain CDRs of SEQ ID NOS: 8, 10 and 12, and light-chain CDRs of SEQ ID NOS: 14, 16 and 18.

19. The method of claim 18, wherein the antibody is antibody scFv having the amino acid sequence of SEQ ID NO: 2.

20. The method of claim 14, wherein the antibody comprises heavy-chain CDRs having homologies of 70% or more with the respective amino acid sequences of SEQ ID NOS: 26, 28 and 30, and light-chain CDRs having homologies of 70% or more with the respective amino acid sequences of SEQ ID NOS: 32, 34 and 36.

21. The method of claim 14, wherein the antibody comprises heavy-chain CDRs of SEQ ID NOS: 26, 28 and 30, and light-chain CDRs of SEQ ID NOS: 32, 34 and 36.

22. The method of claim 21, wherein the antibody is antibody scFv having the amino acid sequence of SEQ ID NO: 20.

23. The method of claim 14, wherein the antibody comprises heavy-chain CDRs having homologies of 70% or more with the respective amino acid sequences of SEQ ID NOS: 69, 71 and 73, and light-chain CDRs having homologies of 70% or more with the respective amino acid sequences of SEQ ID NOS: 75, 77 and 79.

24. The method of claim 14, wherein the antibody comprises heavy-chain CDRs of SEQ ID NOS: 69, 71 and 73, and light-chain CDRs of SEQ ID NOS: 75, 77 and 79.

25. The method of claim 24, wherein the antibody is antibody scFv having the amino acid sequence of SEQ ID NO: 63.

26. The method of claim 1, wherein the intracellular expression comprises introducing into cells a vector comprising a polynucleotide encoding the antibody or an antigen-binding fragment thereof.

27. The method of claim 26, wherein the polynucleotide comprises heavy-chain CDR-encoding nucleotide sequences of SEQ ID NOS: 7, 9 and 11, and light-chain CDR-encoding nucleotide sequences of SEQ ID NOS: 13, 15 and 17.

28. The method of claim 26, wherein the polynucleotide comprises heavy-chain CDR-encoding nucleotide sequences of SEQ ID NOS: 25, 27 and 29, and light-chain CDR-encoding nucleotide sequences of SEQ ID NOS: 31, 33 and 35.

29. The method of claim 26, wherein the polynucleotide comprises heavy-chain CDR-encoding nucleotide sequences of SEQ ID NOS: 68, 70 and 72, and light-chain CDR-encoding nucleotide sequences of SEQ ID NOS: 74, 76 and 78.

30. The method of claim 26, wherein the polynucleotide comprises a nucleotide sequence of SEQ ID NO: 1, 19 or 62.

31. A method of treating or preventing diseases caused by a molecular interaction involving a phosphorylated residue of a phosphorylated protein, the method comprising introducing into a target cell a vector comprising a polynucleotide encoding an antibody that specifically binds to the phosphorylated protein or an antigen-binding fragment thereof.

32. The method of claim 31, wherein the polynucleotide comprises heavy-chain CDR-encoding nucleotide sequences of SEQ ID NOS: 7, 9 and 11, and light-chain CDR-encoding nucleotide sequences of SEQ ID NOS: 13, 15 and 17.

33. The method of claim 31, wherein the polynucleotide comprises heavy-chain CDR-encoding nucleotide sequences of SEQ ID NOS: 25, 27 and 29, and light-chain CDR-encoding nucleotide sequences of SEQ ID NOS: 31, 33 and 35.

34. The method of claim 31, wherein the polynucleotide comprises heavy-chain CDR-encoding nucleotide sequences of SEQ ID NOS: 68, 70 and 72, and light-chain CDR-encoding nucleotide sequences of SEQ ID NOS: 74, 76 and 78.

35. The method of claim 31, wherein the polynucleotide comprises a nucleotide sequence of SEQ ID NO: 1, 19 or 62.

36. An expression system for intracellular expression of an antibody that specifically binds to a phosphorylated protein, comprising a nucleic acid molecule encoding the antibody.

37. The expression system of claim 36, wherein the nucleic acid molecule comprises heavy-chain CDR-encoding nucleotide sequences of SEQ ID NOS: 7, 9 and 11, and light-chain CDR-encoding nucleotide sequences of SEQ ID NOS: 13, 15 and 17.

38. The expression system of claim 36, wherein the nucleic acid molecule comprises heavy-chain CDR-encoding nucleotide sequences of SEQ ID NOS: 25, 27 and 29, and light-chain CDR-encoding nucleotide sequences of SEQ ID NOS: 31, 33 and 35.

39. The expression system of claim 36, wherein the nucleic acid molecule comprises heavy-chain CDR-encoding nucleotide sequences of SEQ ID NOS: 68, 70 and 72, and light-chain CDR-encoding nucleotide sequences of SEQ ID NOS: 74, 76 and 78.

40. The expression system of claim 36, wherein the nucleic acid molecule comprises a nucleotide sequence of SEQ ID NO: 1, 19 or 62.
